(19) United States
(12) Patent Application Publication
(10) Pub. No.: US 2007/0016027 A1
(43) Pub. Date: Jan. 18, 2007

(54) Method and apparatus for utilizing a high speed serial data bus interface within an ultrasound system

(76) Inventors: Gerois Di Marco, St. Francis, WI (US); Snehal Chandrakant Shah, Milwaukee, WI (US); Steven Charles Miller, Waukesha, WI (US); Joseph Kulak, Milwaukee, WI (US)

Correspondence Address: THE SMALL PATENT LAW GROUP LLP, 611 OLIVE STREET, SUITE 1611, ST. LOUIS, MO, 63101, US

(21) Appl. No.: 11/181,343

(22) Filed: Jul. 14, 2005

(51) Int. Cl. A61B 8/00

(52) U.S. Cl. 600/437

(57) ABSTRACT

A medical imaging system is provided. A scan portion is configured to acquire signals. A plurality of interconnected components are configured to receive the signals and communicate information asynchronously.

### BACKGROUND OF THE INVENTION

This invention relates generally to diagnostic imaging systems and methods, and more particularly, to imaging systems and methods configured to transfer imaging information via a high speed serial data bus (HSSDB).

At least some known ultrasound systems experience problems with transmitting large amounts of data from one receiver component to a next receiver component, and ultimately to a radio frequency interface (RFI) and/or host computer of the ultrasound system. More than one beam of imaging data may be constructed substantially simultaneously at the plurality of receiver components. The collecting and processing of echo information along multiple scan lines within a subject is known as multi-line acquisition (MLA). Thus, large amounts of ultrasound information acquired and produced through MLA have to be processed and communicated in the ultrasound system.

Data reduction and/or filtering of data provides one way to accommodate the rapid communication of large amounts of data. Through use of data reduction and/or filtering techniques, non-critical data may be filtered and deleted from the more critical data. However, once data reduction and filtering are performed at the receiver components, the data eliminated cannot be recovered at the RFI or host computer. Some ultrasound applications would be improved by use of all the raw data. For example, the communication of all of the raw imaging data results in producing better quality images. The use of parallel data buses provides another way to communicate large amounts of information rapidly. However, the use of parallel buses typically requires the costly addition of hardware, and synchronicity of the data is near impossible to maintain.

Thus, known methods and systems may not and adequately transmit large amounts of imaging data through a plurality of receiver components and to an RFI and/or host computer of the imaging system.

### BRIEF DESCRIPTION OF THE INVENTION

In one embodiment, a medical imaging system is provided. A scan portion is configured to acquire signals. A plurality of interconnected components are configured to receive the signals and communicate information asynchronously.

In another embodiment, an ultrasound system is provided that includes a plurality of receiver components configured to receive synchronous ultrasound signals and process the received ultrasound signals to output asynchronous ultrasound information. Each of the plurality of receiver components is further configured to receive the asynchronous ultrasound information from at least one other of the plurality of receiver components and combine the asynchronously received ultrasound information with the received synchronous ultrasound signals to output asynchronously combined ultrasound information. The ultrasound system also includes a processor configured to receive asynchronously the combined ultrasound information and process the combined ultrasound information to produce ultrasound image information.

In yet another embodiment, a receiver component for an ultrasound system is provided. The receiver component includes a plurality of inputs configured to receive synchronous ultrasound signals from an ultrasound scan. An interface of the receiver component is configured to receive asynchronous ultrasound information from another receiver component. A processor of the receiver component is configured to combine the received synchronous ultrasound information with the asynchronous received ultrasound information.

In still another embodiment, a method for communicating information within an ultrasound system is provided. The method includes receiving ultrasound signals from an ultrasound scan. Ultrasound information based at least in part on the received ultrasound signals is asynchronously. The method also includes communicating the asynchronous ultrasound information between ultrasound system components. The communicated ultrasound information is combined with the received ultrasound signals to asynchronously provide combined ultrasound information.

### BRIEF DESCRIPTION OF THE DRAWINGS

FIG. 1 is a schematic block diagram of an ultrasound system constructed in accordance with an embodiment of the present invention.

FIG. 2 is a connectivity diagram illustrating connectivity between receiver components and an RFI board the ultrasound system of FIG. 1.

FIG. 3 is a flowchart of an exemplary method for communicating information in an ultrasound system in accordance with an embodiment of the present invention.

FIG. 4 is a block diagram illustrating flow of ultrasound information between a plurality of components in accordance with an embodiment of the present invention.

FIG. 5 is an expanded view of a block diagram illustrating flow of ultrasound information to and from a receiver component in accordance with an embodiment of the present invention.

### DETAILED DESCRIPTION OF THE INVENTION

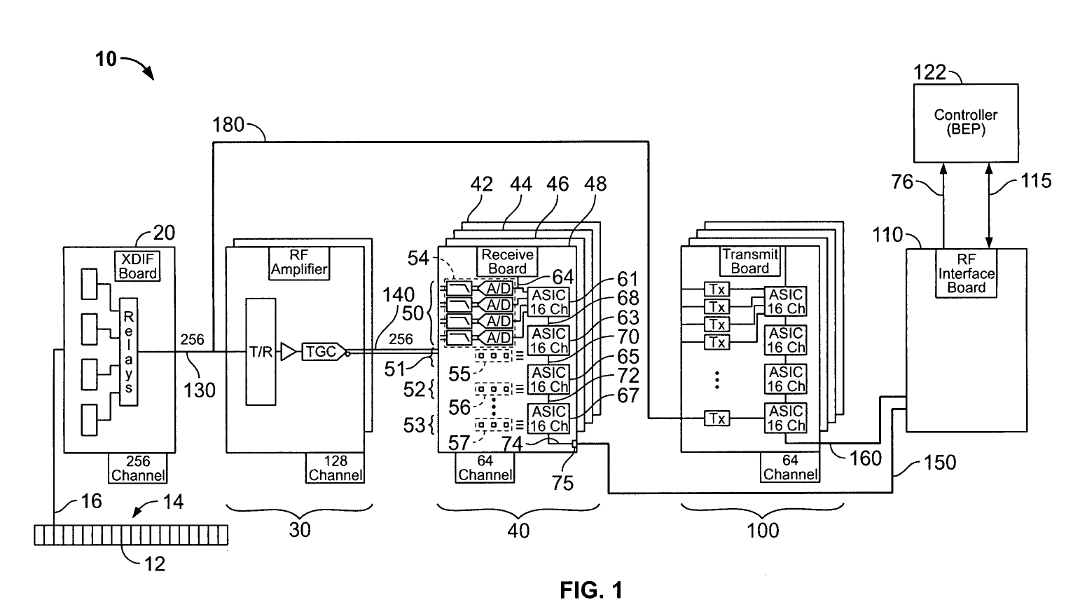

FIG. 1 is a schematic block diagram of an ultrasound system 10 constructed in accordance with an embodiment of the present invention. The ultrasound system 10 generally includes a transducer array 14 having transducer elements 12, a transducer interface board 20, preamplifier boards 30, and a plurality of receiver components 40. Each of the receiver components 40 are identified as a receiver component 42 (e.g., a receiver board), a receiver component 44, a receiver component 46, and a receiver component 48. The ultrasound system 10 also includes a transmit boards group 100 and a Radio Frequency Interface (RFI) board 110. The plurality of receiver components 40, the transmit boards group 100, and the RFI board 110 form a beamformer (BF).

Each of the receiver components 42-48 in the plurality of receiver components 40 has a similar architecture. Thus only one receiver component 48 is described in detail with corresponding structure present in each of the other receiver components. The receiver component 48 includes a plurality of Application Specific Integrated Circuit (ASIC) component groups, namely an ASIC group 50, an ASIC group 51, an ASIC group 52, and an ASIC group 53. Each of the ASIC component groups has a similar architecture, and thus only one ASIC group 50 is described in detail with corresponding structure present in each of the other ASICs. ASIC group 50 includes an A/D converter group 54 and an ASIC 61, the A/D converter group 54 provides inputs 64 to the ASIC 61.

The operator of these components including the flow and processing of information will now be described. The RFI board 110 receives commands from a backend control processor/controller (BEP) 122 over an RFI-BEP controller bus 115 to control the formation of an ultrasound pulse to be emitted into a region of interest (e.g., region of interest of an object to be scanned). The RFI board 110 generates transmit parameters from the received commands that define a transmit beam of a certain shape and size from a certain point or points at the surface of the transducer array 14. The transmit parameters are communicated over a connection 160 (e.g., serial link) from the RFI board 110 to the transmit boards group 100. The transmit boards group 100 generates transmit signals from the received transmit parameters. The transmit signals are provided at certain levels and are phased with respect to each other to steer and focus a transmit beam into one or more transmit pulses or firings.

The transmit boards group 100 communicates the transmit signals via a connection (e.g., communication link) 180 through the transducer interface board 20 to drive the plurality of transducer elements 12 within the transducer array 14 as is known. The connection 180, in one embodiment, includes a plurality of individual channels or communication lines that correspond to the number of transducer elements 12 or to groups of transducer elements 12. The transmit signals excite the transducer elements 12 to emit ultrasound pulses. The ultrasound pulses may be phased to form a focused beam along a desired scan line. Ultrasound echoes, which are backscattered ultrasound waves from, for example, tissue and blood samples within the scanned structure, are received at the transducer elements 12 at different times depending on the distance into the tissue from which the signals are backscattered the angle at which the signals contact the surface of the transducer array 14. The transducer array 14 is a two-way transducer and converts the backscattered waves (ultrasound echoes) of energy into received signals.

The received signals are communicated in separate channels from the transducer array 14 via a connection 16 (e.g., communication links) to the transducer interface board 20, which communicates the received signals over a connection 130 to the preamplifier boards 30. The preamplifier boards 30 perform time gain compensation (TGC) (e.g., swept gain), to increase the amplitude of the received signals from increasing depths in the body to compensate for the progressive attenuation of the deeper echoes. The amplified received signals from the preamplifier boards 30 are communicated over a connection 140 (e.g., communication link) to the plurality of receiver components 40. In the illustrated example, the connections 16, 130, and 140, each include 256 channels and the channels in the connection 140 are divided into four groups of 64 channels. Each of the receiver components 42-48 in the plurality of receiver components 40 receives a group of 64 channels from the preamplifier boards 30.

The group of 64 channels received at receiver component 48 is subdivided into 4 groups of 16 channels. Each group of 16 channels is processed by one of the ASIC groups 50, 51, 52, and 53. For example, a group of 16 channels is received by the A/D converter group 54 of ASIC group 50. The A/D converter group 54 converts the analog signals of the 16 received channels into digital signals providing digital inputs 64 to the ASIC 61. The ASIC 61 processes the received digital signals into beam information and communicates the beam information to a bus 68 then to an ASIC 63. The ASIC group 51 receives a group of 16 channels and uses an A/D converter group 55 to convert the received analog signals into digital signals for use by the ASIC 63. The ASIC 63 processes the received digital signals into processed beam information, and may combine the beam data received over the bus 68 with the processed beam information. The combined beam information is then communicated from the ASIC 63 to an ASIC 65 via a bus 70.

In like manner as described for the ASIC group 51, the ASIC group 52 receives a group of 16 channels at an AID converter group 56. The A/D converter group 56 converts the analog signals of the 16 received channels into digital signals for use by the ASIC 65. The ASIC 65 processes the received digital signals into processed beam information, and may combine the beam data received over the bus 70 with the processed beam data. The combined beam data is then communicated from the ASIC 65 to an ASIC 67 via a bus 72. In similar fashion as described for the ASIC group 52, the ASIC group 53 receives a group of 16 channels at an A/D converter group 57. The A/D converter group 57 converts the analog signals of the 16 received channels into digital signals for use by the ASIC 67. The ASIC 67 processes the received digital signals into processed beam information, and may combine the beam data received over the bus 72 with the processed beam data. The combined beam data is then communicated from the ASIC 67 to a bus 74. The bus 74 communicates the combined beam data from receiver component 48 to a processing chip (e.g., a field programmable gate array (FPGA) 75). The FPGA 75 may combine processed beam data received from another receiver board (e.g. receiver board 46) with the processed beam data received via bus 74. The combined resulting beam data is communicated from FPGA 75 to the RFI board 110 via a high speed serial data bus (HSSDB) 150. The RFI board 110 may further process the received beam data and communicate the resulting processed image information to the BEP 122 over a data bus 76. The BEP 122 may then produce and display ultrasound images from the received image information as is known.

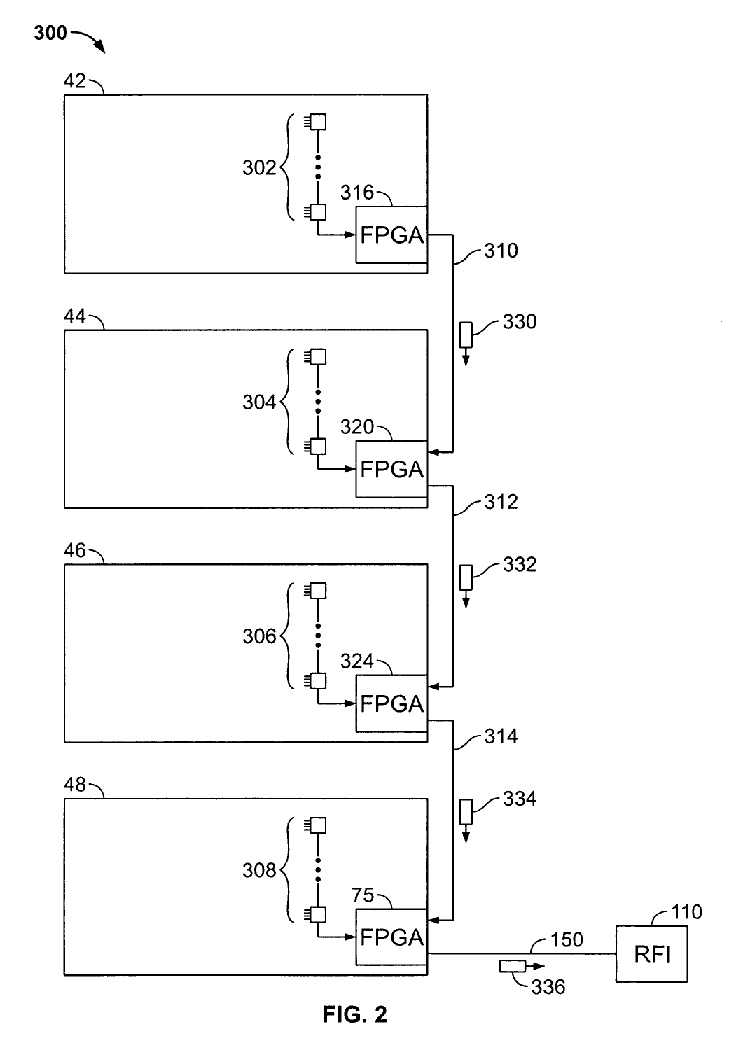

FIG. 2 is a connectivity diagram 300 illustrating connectivity between the receiver components 42-48 of FIG. 1, and between the receiver components 42-48 and the RFI board 110. The exemplary embodiment illustrates a daisy-chained connection (e.g. serial connections) from receiver component 42 to receiver component 44, from receiver component 44 to receiver component 46, from receiver component 46 to receiver component 48, and from receiver component 48 to the RFI board 110. However, it is contemplated that other connection arrangements may be implemented.

Each of the receiver components 42, 44, 46, and 48 has a corresponding group of ASICs, groups 302, 304, 306, and 308, respectively. Each group 302, 304, 306, and 308 receives and processes a set of 64 synchronous signals from the transducer elements 12. Each of the ASICs of the group 302 processes the synchronous signals into beam data and may sum the beam data with beam data from a previous ASIC of the group 302. The resulting beam data from the group 302 is transmitted serially and synchronously to an FPGA 316. The FPGA 316 packetizes the received beam data into a data packet 330 and transmits the data packet 330 asynchronously over an HSSDB 310 to an FPGA 320 of the receiver component 44.

Each of the ASICs of the group 304 processes synchronous signals received by the receiver component 44 into beam data, and may sum the beam data with beam data from a previous ASIC of the group 304. The FPGA 320 unbundles the received data packet 330 of beam data. The FPGA 320 realigns (e.g., re-synchronizes) and interleaves the unbundled beam data with processed beam data received from the group 304 of ASICs. The FPGA 320 packetizes the realigned and interleaved beam data into a data packet 332 and transmits the data packet 332 asynchronously via an HSSDB 312 to an FPGA 324 of the receiver component 46.

Each of the ASICs of the group 306 processes synchronous signals received by the receiver component 46 into beam data, and may sum the beam data with beam data from a previous ASIC of the group 306. The FPGA 324 unbundles the received data packet 332 of beam data. The FPGA 324 realigns (e.g., re-synchronizes) and interleaves the unbundled beam data with processed beam data received from the group 306 of ASICs. The FPGA 324 packetizes the realigned and interleaved beam data into a data packet 334 and transmits the data packet 334 asynchronously via an HSSDB 314 to an FPGA 75 of the receiver component 48.

Each of the ASICs of the group 308 processes synchronous signals received by the receiver component 48 into beam data, and may sum the beam data with beam data from a previous ASIC of the group 308. The FPGA 75 unbundles the received data packet 334 of beam data. The FPGA 75 realigns (e.g., re-synchronizes) and interleaves the unbundled beam data with processed beam data received from the group 308 of ASICs. The FPGA 75 packetizes the realigned and interleaved beam data into a data packet 336 and transmits the data packet 336 asynchronously via an HSSDB 150 to an FPGA 75 of the RFI 110.

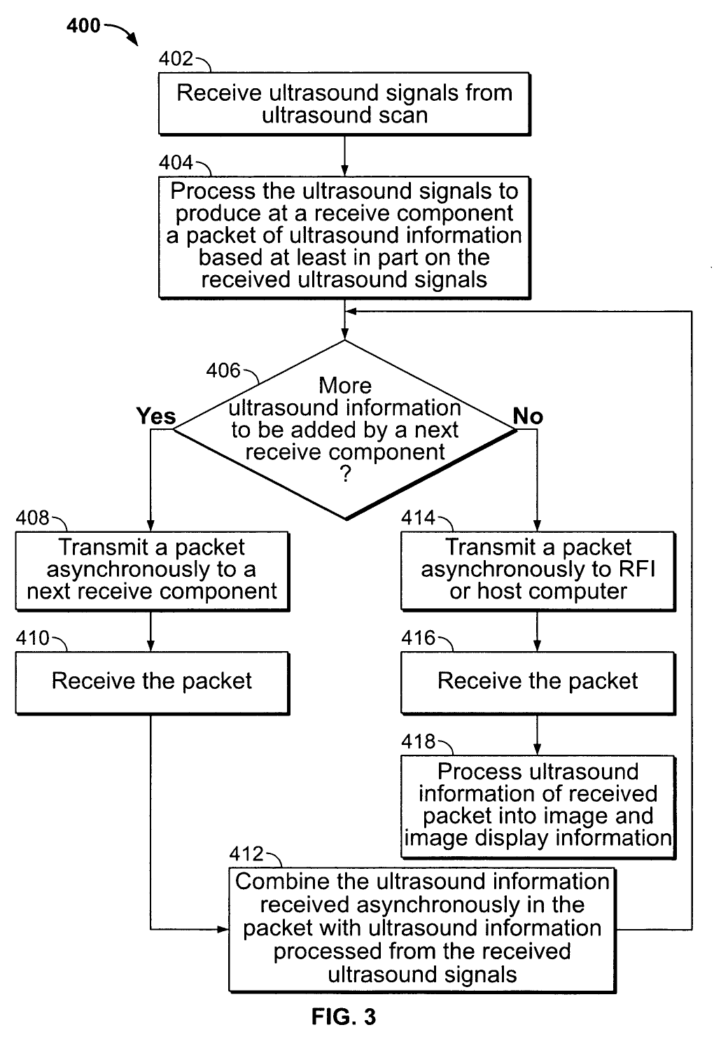

FIG. 3 is a flowchart of an exemplary method 400 for communicating information in an ultrasound system 10 in accordance with an embodiment of the present invention. The technical effect of the method 400 is the asynchronous communication of ultrasound information between components of the ultrasound system. The method 400 generally provides for receiving ultrasound signals from an ultrasound scan and producing a packet of ultrasound information based in part on the received ultrasound signals. The packet is asynchronously transmitted at high speed. When received, the packet of ultrasound information may be combined with the received ultrasound signals to produce asynchronously a packet of combined ultrasound information. The technical effect produces packets of ultrasound information that are transmitted asynchronously. The packet may be transmitted over a high speed serial data bus (HSSDB) at a rate of, for example, approximately 1 gigabyte per second (Gbps), or 1.5 Gbps, or greater than 1.5 Gbps. The technical effect is achieved by performing the method 400 described in more detail below.

Specifically, using the method 400, synchronous ultrasound signals from an ultrasound scan (e.g. inputs 64 shown in FIG. 1) are received 402. At 404, a data packet of ultrasound information is produced at a receiver component based at least in part on the received ultrasound signals. For example, the group 302 of ASICs (shown in FIG. 2) receive synchronous input signals and process the signals into ultrasound information. The ultrasound information is communicated from the group 302 (shown in FIG. 2) to the FPGA 316 (shown in FIG. 2). The FPGA 316 produces a packet 330 containing the ultrasound information. The producing 404 of the data packet 330 may include concurrently transmitting the packet 330 while receiving the ultrasound signals to produce the ultrasound information for inclusion in the packet 330. Producing 404 the packet 330 may further include providing error check information in a trailer of the packet 330. At 406, a determination is made as to whether more ultrasound information is to be added to the packet by a next receiver component. If more ultrasound information is to be added, the packet 330 is transmitted asynchronously at 408 to the next receiver component (e.g., receiver component 44 shown in FIG. 2).

The next receiver component (e.g., the receiver component 44) receives at 410 the packet 330. At 412, the ultrasound information received asynchronously in the packet 330 is combined with ultrasound information processed from the synchronous ultrasound signals received at receiver component 44 to produce a packet (e.g., the packet 332) of combined ultrasound information. For example, the group 304 of ASICs receive synchronous input signals and processes the signals into ultrasound information. The ultrasound information is communicated to the FPGA 320 for packetizing. The combining 412 further may include realigning the ultrasound information from the asynchronously received packet 330 with the processed ultrasound information for inclusion in the packet 332. The combined ultrasound information may include inserted idles, as described herein. Processing flow then returns to the determination at 406.

If more ultrasound information is to be added by a next receiver component (e.g. the receiver component 46 or 48 of FIG. 2), the processing at 408, 410, and 412 is repeated. If not, the packet of combined ultrasound information produced at 412 is transmitted at 414 to the RFI 110 and/or to the host computer 122 (shown in FIG. 1). The packet is received at 416, and processed at 418 by the RFI 110 and/or the host computer 122. The processing at 418 results in image information that may be displayed as an ultrasound image.

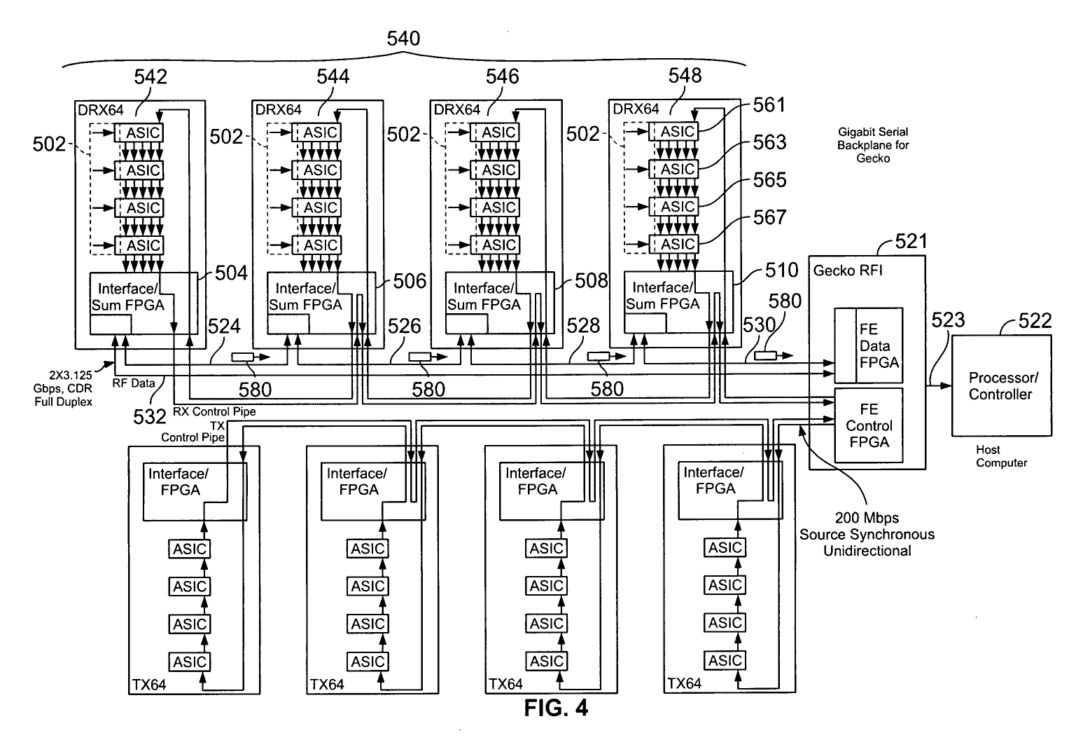

FIG. 4 is a block diagram illustrating flow of ultrasound information between a plurality of receiver components 540 (e.g., receiver components 542, 544, 546, and 548) in accordance with an embodiment of the present invention. Each of the plurality of receiver components 540 may be substantially the same. Each of the plurality of receiver components 540 is configured to receive synchronous ultrasound signals 502 from the transducer elements 12 (shown in FIG. 1) and process the received ultrasound signals 502 to transmit asynchronously ultrasound information 580 using the methods described in connection with FIG. 3. Each of the plurality of receiver components 540 is further configured to receive 410 asynchronously ultrasound information 580 from at least one other of the plurality of receiver components 540. The asynchronously received ultrasound information 580 is combined 412 with ultrasound information processed from the received synchronous ultrasound signals 502 to produce combined ultrasound information 580. Each receiver component 542-548 is further configured to produce 404 a continuous stream of asynchronous packets of ultrasound information 580.

Each of the plurality of receiver components 540 is configured to transmit asynchronously a packet of the ultrasound information 580 concurrently with receiving the ultrasound signals 502 to produce the ultrasound information 580 for inclusion in the packet. The transceivers 504, 506, 508 and 510 are configured to communicate ultrasound information 580 bi-directionally and may include FPGAs. An RFI 521 is configured to receive asynchronously the combined ultrasound information 580. The RFI 521 processes the combined ultrasound information 580 to produce ultrasound image information. The ultrasound image information is transmitted via a bus 523 to a host computer/controller 522 for use in producing an ultrasound image. Although the RFI 521 and the host computer 522 are shown in FIG. 4 as distinct separate components, in an alternative embodiment, a host computer with the combined functionality may be provided instead of the RFI 521 and the host computer 522.

The processing of ultrasound signals 502 into ultrasound information at a receiver component 542-548 may be performed by a group of ASICs, (e.g., ASICs 561, 563, 565, and 567) of the receiver component 548. In an alternative embodiment, a group of processing elements other than ASICs may be used to perform the same functionality. The processing of the ultrasound information produced by a group of ASICs to form packets of ultrasound information 580 may performed by an FPGA transceiver (e.g., transceiver 510). In an alternative embodiment, a processing element other than an FPGA may be used in the transceivers 504-510. FIG. 4 shows four receiver components 542-548. However, in an alternative embodiment, either one or a plurality of interconnected receiver components may be provided. HSSDBs 524, 526, 528, and 530 are shown as serially connecting (daisy-chaining) the receiver components 542-548 and the RFI 521. In an alternative embodiment, the HSSDBs 524-530 may connect to the RFI 521 in a parallel arrangement. The HSSDBs 524-530 may provide uni-directional flow of the ultrasound information 580 from the receiver components 542-548 to the RFI 521. In an alternative embodiment, the HSSDBs 524-530 and a HSSDB 532 may provide bi-directional flow of the ultrasound information 580 between the receiver components 542-548 and the RFI 521.

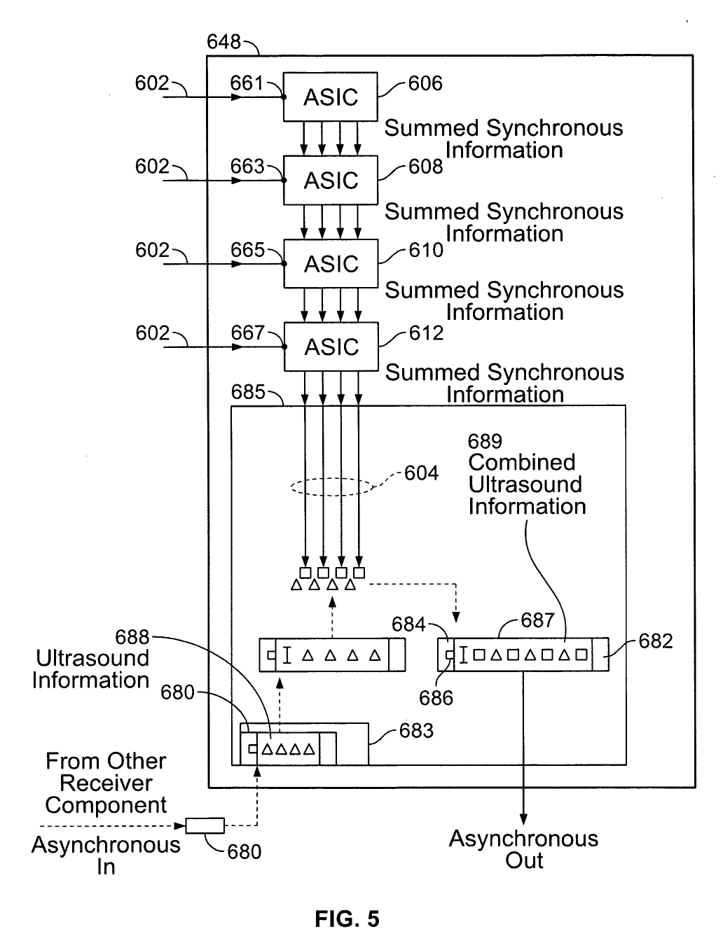

FIG. 5 is an expanded view of a flow of ultrasound information to and from a receiver component 648 in accordance with an embodiment of the present invention. In the embodiment of FIG. 5, the receiver component 648 may be, for example, a receiver circuit board. The receiver component 648 includes a plurality of inputs 661, 663, 665, and 667 configured to receive ultrasound signals 602 from an ultrasound scan. The receiver component 648 includes an interface 683 configured to receive asynchronously a data packet 680 containing ultrasound information 688 from another receiver component. The receiver component 648 also includes a processor 685 configured to combine the received synchronous ultrasound information 604 with the asynchronously received ultrasound information 688 as described herein. The processor 685 is configured to unbundle the incoming asynchronous data packet 680 received from another receiver component and combine/interleave the unbundled ultrasound information 688 with the synchronous ultrasound information 604 to produce the combined ultrasound information 689. Processed ultrasound information is received from ASICs 606, 608, 610, and 612 to produce the ultrasound information 604. The combined ultrasound information 689 is bundled into a data packet 687. A lapse in the synchronous ultrasound information 604 results in one or more idles being inserted as a spacer in the rebundled packet 687. An idle is, for example, a filler with no ultrasound information included. The data packets 680 and 687 may be variable in length. Each data packet, (e.g., the packet 687) includes a header 682, the ultrasound information 689, and a trailer 684. The trailer 684 signals the end of the bundling of the packet. The trailer 684 further includes an error check field 686. The error check field 686 is in the trailer 684 of the data packet 687 due to the packet 687 being filled with synchronous ultrasound information 604 concurrently with the packet 687 being transmitted.

While the invention has been described in terms of various specific embodiments, those skilled in the art will recognize that the invention can be practiced with modification within the spirit and scope of the claims.

What is claimed is:

1. A medical imaging system, comprising:
a scan portion configured to acquire signals; and
a plurality of interconnected components configured to receive the signals and communicate information asynchronously.

2. A medical imaging system in accordance with claim 1, wherein the signals comprise synchronous signals.

3. A medical imaging system in accordance with claim 1, wherein the interconnected components serially convey data bidirectionally.

4. A medical imaging system in accordance with claim 1, wherein the information comprises data packets.

5. A medical imaging system in accordance with claim 1, wherein the information is communicated at a serial data rate of at least 1 gigabit per second (Gbps).

6. A method for communicating information within an ultrasound system, said method comprising:
receiving ultrasound signals from an ultrasound scan;
generating asynchronously ultrasound information based at least in part on the received ultrasound signals;
communicating the asynchronously ultrasound information between ultrasound system components; and
combining the communicated ultrasound information with the received ultrasound signals to asynchronously provide combined ultrasound information.

7. A method in accordance with claim 6, wherein said combining further comprises realigning the asynchronous ultrasound information with the received ultrasound signals.

8. A method in accordance with claim 6, wherein said asynchronously providing further comprises concurrently transmitting the asynchronous information while receiving the ultrasound signals to generate additional asynchronous ultrasound information.

9. A method in accordance with claim 6, wherein said producing asynchronously a packet further comprises including error check information in a trailer of the packet.

10. A method in accordance with claim 6, further comprising processing the combined ultrasound information to produce ultrasound image information.

11. A method in accordance with claim 6, wherein the received ultrasound signals comprise synchronous signals.

12. An ultrasound system, comprising:
a plurality of receiver components configured to receive synchronous ultrasound signals and process the received ultrasound signals to output asynchronous ultrasound information, each of the plurality of receiver components further configured to receive asynchronous ultrasound information from at least one other of the plurality of receiver components and combine the asynchronously received ultrasound information with the received synchronous ultrasound signals to output asynchronously combined ultrasound information; and
a processor configured to receive asynchronously the combined ultrasound information and process the combined ultrasound information to produce ultrasound image information.

13. An ultrasound system in accordance with claim 12, wherein the ultrasound information comprises data packets.

14. An ultrasound system in accordance with claim 12, wherein the ultrasound information comprises data packets, each of the data packets comprising an error check field in a trailer of the data packet.

15. An ultrasound system in accordance with claim 12, wherein each of the plurality of receiver components is configured to realign the asynchronously received ultrasound information with the received synchronous ultrasound signals to produce asynchronously the combined ultrasound information.

16. An ultrasound system in accordance with claim 12, wherein each of the plurality of receiver components is configured to transmit asynchronously a packet of the ultrasound information concurrently with receiving the ultrasound signals to produce the ultrasound information for inclusion in the packet.

17. An ultrasound system in accordance with claim 12, wherein the combined ultrasound information further includes inserted idles.

18. An ultrasound system in accordance with claim 12, wherein each of said plurality of receiver components is further configured to produce a continuous stream of asynchronous packets of ultrasound information.

19. An ultrasound system in accordance with claim 12, wherein each of said plurality of receiver components comprises an ultrasound receiver board.

20. A receiver component for an ultrasound system, said receiver component comprising:
a plurality of inputs configured to receive synchronous ultrasound signals from an ultrasound scan;
an interface configured to receive asynchronous ultrasound information from another receiver component; and
a processor configured to combine the received synchronous ultrasound information with the asynchronously received ultrasound information.
